(19) United States
(12) Patent Application Publication
(10) Pub. No.: US 2005/0205685 A1
(43) Pub. Date: Sep. 22, 2005

(54) Dispenser

(76) Inventor: Stuart Michael Ruan Jones, Herts (GB)

Correspondence Address: NORRIS, MCLAUGHLIN & MARCUS, 875 THIRD AVE, 18TH FLOOR, NEW YORK, NY, 10022, US

(21) Appl. No.: 10/521,184

(22) PCT Filed: Jul. 8, 2003
(86) PCT No.: PCT/GB03/02916

§ 371 Date: May 13, 2005

(30) Foreign Application Priority Data
Jul. 17, 2002 (GB) 0216556.1

(51) Int. Cl. A61L 9/04; A24F 25/00

(52) U.S. Cl. 239/43; 239/53; 239/55

(57) ABSTRACT

A portable device for dispensing a fragrance comprising a housing (1) containing a fragrance element (3) and at least one aperture (5) in a side of the housing (1) in the vicinity of one end (6) of the housing to release fragrance from the housing (1). A lid is attached to the housing (1) to selectively open and close the aperture (5). The one end (6) of the device is shaped to abut the lower end of a users nose such that in use the fragrance can be inhaled through the aperture (5) to the nasal passage of the user.

The present invention relates to a portable device for dispensing a fragrance.

In everyday life we are subjected to offending olfactory sensations. For example, cooking smells, cigarette smoke and stale air when we are confined to closed environments, such as trains, cars and houses with limited air exchanging properties. Such strong smells are capable of causing a person to faint or to cause them considerable discomfort. Accordingly, occasions arise when a portable air freshening device would be desirable.

It is known to use smelling-bottles. These bottles are portable and contain smelling salts such as ammonium carbonate mixed with a scent to be sniffed as a restorative in faintness.

Furthermore, inhalers are known which comprise a stick with a vent at the extremity of the device and apertures around the base of the device. In use, the inhaler is inserted into the nostril of a user. The user then holds the other nostril closed and inhales deeply. The apertures at the base of the stick allow air flow through the inhaler which causes evaporation of a decongestant which is subsequently inhaled through the vent at the extremity of the stick.

Such devices are designed to deliver an intense amount of the inhaled product to the nasal passages and are therefore unsuitable for delivering certain fragrances.

Accordingly, the present invention provides a portable device for dispensing a fragrance comprising:
a housing containing a fragrance element;
at least one aperture in a side of the housing in the vicinity of one end of the housing to release the fragrance from the housing;
and means to selectively open and close the aperture;
the one end being shaped to abut the lower end of a users nose such that in use the fragrance can be inhaled through the aperture to the nasal passage of a user.

The device of the present invention is advantageous over the prior art in that the relationship between the one end and the aperture is such that the aperture is close to, but not directly in contact with the users nose. Thus, the fragrance can be provided in a lower intensity whilst still guaranteeing local delivery to the user. This ensures that the fragrance does not affect people in the vicinity of the user.

Any composition which can release a pleasant smell and is suitable for use as a fragrance can be used in the present invention. Beyond the subjective enjoyment produced by pleasant fragrances, certain fragrances may produce psychological and physiological responses such as stress reduction due to activation of brain centres which are especially responsive to fragrances. In addition to fragrances, humans may enjoy and benefit from inhaling volatile substances which may not have a detectable scent, but which none-the less produce a distinct biological or psychological effect. These substances include, but are not limited to pheromones which may alter the sexual behavior of humans, hormones which may alter the physiology of the body, mood altering substances, appetite-altering substances, organ extracts, plant extracts or other materials or chemicals which provide a desired biological or psychological effect. The word “fragrance” in the context of the present invention encompasses all of the aforementioned substances.

The fragrance element may be any fragrance in any form which is vaporizable at room temperature and atmospheric pressure. For example the fragrance element may be prepared as a gel or a paste to control spills or leaks. Furthermore, the fragrance element may be in liquid form and placed inside a reservoir. In a preferred embodiment a porous solid such as scintered PE or cellulose board is impregnated with the fragrance.

A fragrance element is placed in the enclosure formed by the housing. The fragrance element can be introduced into the enclosure at anytime during the assembly of the housing. In a preferred embodiment the fragrance element may have as a component of its design a form to assist in locations and attachment of the fastening element to the housing to secure it. For example one or more anchor pins, shafts or posts can be present.

Furthermore, in a preferred embodiment the device may have as part of its design sections of the housing which can be opened and/or removed to allow the user to replace and/or replenish the fragrance element so that the device can be used again.

The number of apertures present within the side of the housing in the vicinity of one end of the device is dependent upon the size of the device, the diameters of the apertures and the fragrance release rate required. Desirably the number of apertures is from 1 to 20, more preferably 5 to 15 and most preferably 10. The average diameter of each aperture is preferably from 1 mm to 7 mm, more preferably from 1 to 4 mm. The apertures of the device can have all the same diameter but in a preferred embodiment a plurality of apertures are present with varying diameters. The apertures may be arranged in an orderly or random pattern.

Any means can be used to selectively open and close the apertures. For example a stopper or a seal. In a preferred embodiment the means to selectively open and close the apertures is lid. The lid can be pivotally attached to the housing wherein the lid is movable from a closed position where the lid covers the aperture to seal the device to an open position where the aperture is uncovered. Desirably the lid is integrally hinged to the housing.

In a preferred embodiment the lid in its open position rests against the side of the housing adjacent to the aperture such that in use the lid prevents the aperture from the device from coming into contact with the top lip of the user.

In a preferred embodiment a catch is present which is positioned in the one end of the device to provide the user with a clear indication of the successful opening and closing of the lid by providing an audible “click”. The “click” is provided by the interaction of the lid as it passes over the catch.

The one end of the device is arranged to a but the lower end of the nose. In a preferred embodiment the one end has a central recess to accommodate the septum of the user.

For a better understanding of the invention it will now be described, by way of example, with reference to the accompanying drawings, in which:

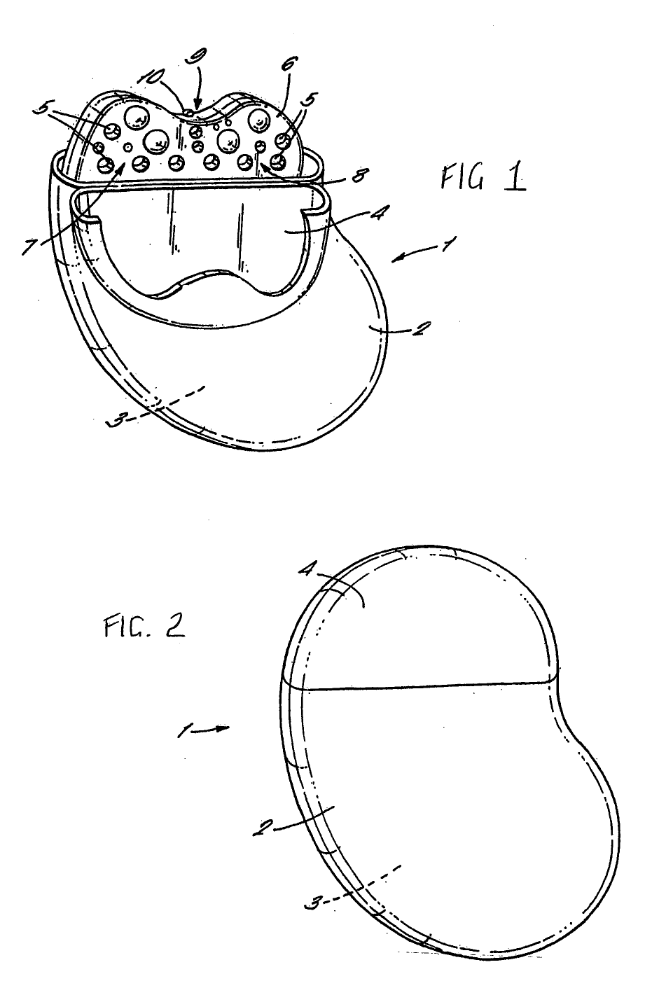

FIG. 1 is a perspective view of the device with the lid open;

FIG. 2 is a perspective view of the device with the lid closed.

As shown in FIGS. 1 and 2 the device is in the shape of a flattened kidney bean. Such a shape enables the device to be held comfortably in the hand.

The device is typically about 70 mm long and about 45 mm across.

The housing 1 is made of plastic. The housing forms an inner enclosure 2 which contains the fragrance element 3.

The device further comprises a lid 4 which is pivotally attached to a side of the housing. The lid 4 is also made of plastic. The lid 4 is of such a shape that when the lid 4 is in its closed position it covers the apertures 5 to seal the device and is flush with the housing, as shown in FIG. 2.

Furthermore, when the lid 4 is in its open position it can rest against the side of the housing, as shown in FIG. 1, such that in use the lid 4 prevents the apertures 5 of the device coming into contact with the skin, and in particular the top lip of the user.

The section of the side of the housing which is covered by the lid 4 has a plurality of apertures 5 in the vicinity of the end 6 of the device. The apertures 5 are arranged in a random pattern. There are 11 apertures present. The diameters of the apertures 5 vary from 1.5 to 4 mm.

The end 6 of the device is arranged to abut the lower end of the nose. A first end region 7 is adapted to abut a first nasal passage and a second end region 8 is adapted to abut a second nasal passage with central recess 9, between the first and second end regions 7,8 to accommodate the septum.

As shown in FIG. 1 there is a catch 10 positioned in the central recess 9 which holds the lid 4 in place when closed and provides an audible “click” when the lid 4 passes over the catch 10.

In use the user will open the lid 4 of the device, which will be signified to the user by a “click” as the lid passes over the catch 10, and then position the end of the device Z so that it abuts the lower end of the nose.

As illustrated, the device of the present invention does not include powered means for dispensing the fragrance. Rather, the device utilizes air flow created by the user sniffing the housing 1 to promote a flow of air saturated with fragrance from the fragrance element 3 through the apertures 5 to the nasal passages of the user.
After use, the lid 4 will be shut, which is signified to the user by a “click” as the lid 4 passes over the catch 10, to ensure that the apertures 5 are covered and that the fragrance is selected within the device. This allows the air in the enclosure to become saturated with the fragrance.

1. A portable device for dispensing a fragrance comprising:
a housing containing a fragrance element;
at least one aperture in a side of the housing in the vicinity of one end of the housing to release the fragrance from the housing;
and means to selectively open and close the aperture which is a lid pivotally attached to the housing wherein the lid is movable from a closed position where the lid covers the aperture to seal the device to an open position where the aperture is uncovered;
the one end being shaped to abut the lower end of a users nose such that in use the fragrance can be inhaled through the aperture to the nasal passage of the user.

2. A device according to claim 1 wherein the lid in its open position rests against the side of the housing adjacent to the aperture such that in use the lid prevents the aperture of the device from coming into contact with the top lip of the user.

3. A device according to claim 1 wherein the one end has a catch to provide an audible “click” as the lid passes over the catch.

4. A device according to claim 1 wherein the one end has a central recess to accommodate the septum of the user.
